(19) United States
(12) Patent Application Publication
(10) Pub. No.: US 2003/0149322 A1
(43) Pub. Date: Aug. 7, 2003

(54) Process of producing C2 and C3 olefins from hydrocarbons

(76) Inventors: Ulrich Koss, Darmstadt (DE); Peter Konig, Bad Homburg (DE); Martin Rothaemel, Frankfurt am Main (DE)

Correspondence Address: Kurt G Briscoe, Norris Mclaughlin & Marcus, 30th Floor, 220 East 42nd Street, New York, NY, 10017, US

(21) Appl. No.: 10/169,703

(22) PCT Filed: Jan. 8, 2001
(86) PCT No.: PCT/EP01/00129

(30) Foreign Application Priority Data
Jan. 12, 2000 (DE) 10000889.5

(51) Int. Cl. C07C 4/04; C07C 4/02

(52) U.S. Cl. 585/652; 585/650

(57) ABSTRACT

The feed hydrocarbon together with steam is passed as vapor through a thermal steam cracking in which it is heated to temperatures in the range from 700 to 1000° C., where a cracking mixture is produced which contains C₂- to C₆-olefins and C₄- to C₆-diolefins. From the cracking mixture a first fraction, which contains C₂- and C₃-olefins, and a second fraction, which contains olefins and diolefins of the range C₄ to C₆, are separated. The diolefins are at least partly removed from the second fraction, and an intermediate product is produced which consists of C₄- to C₆-olefins for at least 30 wt-%. A feed mixture containing C₄- to C₆-olefins and steam is introduced into a reactor with an inlet temperature of 300 to 700° C., which reactor contains a bed of granular, form-selective catalyst, where a product mixture containing C₂- to C₄-olefins is withdrawn from the bed and C₂- and C₃-olefins are separated from the product mixture.

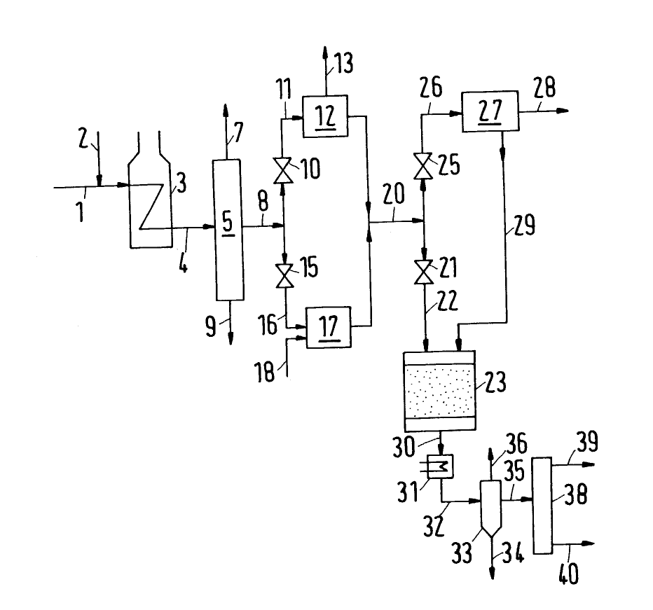

### DESCRIPTION

This invention relates to a process of producing C₂- and C₃-olefins from hydrocarbons.

Such process is known from U.S. Pat. No. 5,981,819, which proceeds from a feed hydrocarbon containing C₄- to C₇-olefins, which hydrocarbon is reacted on a form-selective zeolite catalyst.

It is the object underlying the invention to develop the known process and to be able to employ a hydrocarbon mixture obtained as intermediate product, which also has higher-boiling components. In accordance with the invention this is achieved in that the feed hydrocarbon together with steam is passed as vapor through a thermal steam cracking in which it is heated to temperatures in the range from 700 to 1000° C., where a cracking mixture is produced which contains C₂- to C₆-olefins and C₄- to C₆-diolefins. From the cracking mixture a first fraction, which contains C₂- and C₃-olefins, and a second fraction, which contains olefins and diolefins of the range C₄ to C₆, are separated. The second fraction may for instance only contain C₄-olefins or in addition C₄-diolefins, and it may for instance only consist of C₅- and C₆-olefins and C₅- and C₆-diolefins. From the second fraction, the diolefins are at least partly removed and an intermediate product is produced, which consists of C₄- to C₆-olefins for at least 30 wt-%, that a feed mixture containing C₄- to C₆-olefins and steam is introduced into a reactor with an inlet temperature of 300 to 700° C., which reactor contains a bed of granular, form-selective catalyst, where a product mixture containing C₂- to C₄-olefins is withdrawn from the bed and C₂- and C₃-olefins are separated from the product mixture. The hydrocarbon mixture introduced into the steam cracking for instance is naphtha or ethane.

From the second fraction, which contains olefins and diolefins of the range C₄ to C₆ and is separated from the product of the steam cracking, the diolefins (e.g. butadiene, pentadiene, hexadiene) must first be separated to a residual content of preferably not more than 5 wt-%. This is necessary because the diolefins disturb the further treatment, as they may contribute to a rapid carbonization of the form-selective catalyst. For removing the diolefins from the second fraction, several possibilities exist, and they may for instance be removed extractively or they are at least partly converted to olefins (e.g. butene, pentene, hexene) by a partial hydrogenation.

It may be expedient to wholly or partly use the mixture at least partly liberated from diolefins, here referred to as intermediate product, for producing methyl-tert-butylether (MTBE). For this purpose, at least part of the intermediate product may be passed through an MTBE synthesis, where in particular the isobutene contained therein is-converted to MTBE on a catalyst known per se by adding methanol. Details of the MTBE synthesis are known (for example process of Snamprogetti or Universal Oil Products).

A feed mixture containing steam and C₄- to C₆-olefins is finally passed over a granular, form-selective zeolite catalyst. The zeolite preferably is of the pentasil type with an atomic ratio Si:Al in the range from 10:1 to 200:1. Such zeolite catalyst is described for instance in EP-B-0369364. It is recommended, to operate the reactor which contains the zeolite catalyst at relatively low pressures in the range from 0.2 to 3 bar and preferably 0.6 to 1.5 bar. Details are known from U.S. Pat. No. 5,981,819.

Embodiments of the process will be explained with reference to the drawing. The drawing shows a flow diagram of the process.

A vaporous feed hydrocarbon, which may also be a hydrocarbon mixture, e.g. naphtha, is supplied via line 1, mixed with steam from line 2 and passed through a steam cracker 3. The steam cracker is heated in a manner known per se by burning a fuel, where by an indirect heat exchange the mixture to be cracked is briefly heated to temperatures in the range from 700 to 1000° C. Under these conditions, larger molecules are thermally cracked. Via line 4, a cracking mixture is withdrawn, which usually contains C₂- to C₂₀-olefins and also higher-boiling components. In a distillation 5, which may also have a multi-stage configuration, the desired fractions are separated from the mixture supplied. A first fraction, which contains C₂- to C₃-olefins, is discharged via line 7 and already represents a raw product. A second fraction, which contains olefins of the range C₄ to C₆, is withdrawn via line 8, and the heavier components are obtained in line 9.

To at least partly remove diolefins and in particular butadiene from the second fraction of line 8, two possibilities are represented in the drawing, which may also be utilized at the same time. The first possibility is to pass through the open valve 10 and line 11 to an extraction 12 in which butadiene is removed. This extraction operates in a manner known per se, for instance according to a process licensed by the firm BASF. Extracted butadiene is discharged through line 13.

The second possibility for the further treatment consists in that the second fraction of line 8 is wholly or partly charged through the open valve 15 and line 16 to a hydrogenation 17, to which hydrogen gas is also supplied through line 18. In the hydrogenation, which operates catalytically in a manner known per se, diolefins are at least partly converted to olefins. The product of the hydrogenation 17 and the mixture from the extraction 12 are combined in line 20 and there is formed a mixture which here is referred to as intermediate product. This intermediate product consists of C₄- to C₆-olefins for at least 30 wt-% and preferably at least 50 wt-%.

It is easily possible that the intermediate product of line 20 is wholly or only partly charged through line 22 to a reactor 23, in order to produce the desired C₂- and C₃-olefins. One process variant consists in that the intermediate product of line 20 is wholly or partly supplied through the open valve 25 and line 26 to an MTBE synthesis 27. By means of this synthesis, which operates in a manner known per se, MTBE is recovered, which is used as anti-knocking agent in fuels for prime movers. MTBE is withdrawn via line 28. The remaining gas mixture likewise reaches the reactor 23 through line 29.

The reactor 23 contains a bed of a granular, form-selective zeolite catalyst. At temperatures of 300 to 700° C. in the bed the feed material supplied via lines 22 and 29 is largely converted to C₂- and C₃-olefins.

The product mixture coming from the reactor 23 is withdrawn via line 30 and cooled to temperatures of about 30 to 80° C. in a cooler 31, so that water and gasoline will condense out. The condensate-containing mixture flows through line 32 to a separator 33. From the separator, water is withdrawn through line 34, in line 35 an organic gas phase is obtained, and through line 36 a product gas is withdrawn. The product gas contains the desired products ethylene and propylene. To separate the valuable substances ethylene and propylene, the gas of line 36 can be supplied to a separating means not represented.

The organic gas phase 35 is partly condensed in the distillation column 38 and divided into a gaseous phase containing C₄-olefins, which gaseous phase is withdrawn through line 39, and into a liquid phase, which is withdrawn through line 40.

### EXAMPLES

There is employed a plant corresponding to the drawing, and 89 t/h naphtha, 6 t/h ethane and 42 t/h steam are supplied to the steam cracker 3. The data of the examples have been calculated in part, all compositions (in wt-%) are indicated without the steam content. The cracking mixture leaving the steam cracker via line 4 with a temperature of 380° C. has the composition indicated in Table I, column A (in wt-%):

TABLE I

| | A | B | C | D | E | F | G |
|---|---|---|---|---|---|---|---|
| Diolefins | 6.6 | — | 35.6 | 0.5 | — | — | 0.8 |
| Olefins: | | | | | | | |
| Ethylene | 28.9 | 52.6 | — | — | 18.5 | — | — |
| Propylene | 16.5 | 30.8 | — | 0.1 | 78.4 | — | 0.1 |
| 1-butene | 1.6 | — | 15.5 | 24.0 | — | 8.7 | 42.7 |
| Iso-butene | 3.0 | — | 28.6 | 44.2 | — | 30.1 | 0.6 |
| 2-butene | 0.8 | — | 7.7 | 11.9 | — | 28.0 | 21.2 |
| Pentene | 0.8 | — | — | 0.1 | — | — | 0.1 |
| Paraffins | 8.2 | 14.7 | 12.6 | 19.2 | 2.7 | 31.2 | 34.5 |
| Aromatics and | 32.5 | — | — | — | — | — | — |
| naphthenes | | | | | | | |
| H₂ | 1.1 | 1.9 | — | — | 0.4 | — | — |

Upon cooling and fractionating there is obtained a first fraction with the composition indicated in Table I, column B, and a second fraction with the composition indicated in column C. This second fraction is further processed in various ways, which are described in Examples 1 to 4:

### Example 1

With the valve 15 closed, the second fraction is supplied through line 11 to a butadiene extraction 12 known per se, and in line 20 an intermediate product with the composition in accordance with Table I, column D is obtained. This intermediate product is charged into the reactor 23 with an inlet temperature of 500° C. and with a weight ratio H₂O: hydrocarbons of 1.8:1, the zeolite catalyst is described in U.S. Pat. No. 5,981,819 (Examples). In line 36, a product fraction with the composition in accordance with Table I, column E is obtained, which together with the first fraction (Table I, column B) is passed through a gas separation plant to recover the end products ethylene and propylene in the desired purity. A second product fraction, which is obtained in line 39 and has the composition in accordance with Table I, column F. will be added to the cracking mixture of line 4, whereby the yield of ethylene and propylene can be increased.

### Example 2

The procedure is as in Example 1, but with the valve 21 closed the intermediate product of line 20 is supplied through line 26 to an MTBE synthesis 27 known per se, where isobutylene is reacted with methanol to form MTBE, and this product is withdrawn through line 28. The remaining mixture, which flows to the reactor 23 via line 29, has the composition indicated in Table I, column G. The reaction in the reactor 23 is effected under the same conditions as in Example 1, which is also true for the subsequent separation of ethylene and propylene.

### Example 3

A second fraction with the composition indicated in Table II, column A (in wt-%) is obtained in line 8.

TABLE II

| | A | B | C | D | E | F | G |
|---|---|---|---|---|---|---|---|
| Diolefins | 35.5 | — | — | — | — | — | — |
| Olefins: | | | | | | | |
| Ethylene | — | — | 18.7 | — | — | 18.5 | — |
| Propylene | — | — | 79.0 | — | 0.1 | 78.4 | — |
| 1-butene | 15.6 | 31.6 | — | 9.6 | 43.8 | — | 8.8 |
| Iso-butene | 28.6 | 28.3 | — | 33.2 | 0.6 | — | 30.1 |
| 2-butene | 7.7 | 26.6 | — | 30.9 | 36.8 | — | 28.0 |
| Pentene | — | 0.2 | — | — | 0.3 | — | — |
| Paraffins | 12.6 | 13.3 | 2.0 | 26.3 | 18.4 | 2.7 | 33.1 |
| Other substances | — | — | 0.3 | — | — | 0.4 | — |

With the valve 10 closed, this second fraction together with hydrogen from line 18 is supplied through line 16 to a partial hydrogenation 17 on a commercially available Pd/Al₂O₃ catalyst disposed in a fixed bed. The mixture withdrawn from the hydrogenation has the composition indicated in Table II, column B, and it is supplied to the reactor 23 through lines 20 and 21. The procedure takes place in the reactor 23 and subsequently as shown in Example 1, the product stream of line 36 has the composition in accordance with Table II, column C, and column D indicates the composition of the gas mixture of line 39.

### Example 4

The procedure first of all takes place with the valve 10 closed, as in Example 3, the valve 21 is now also kept closed, and the intermediate product of line 20—Table II, column B—is supplied to the MTBE synthesis 27. Upon separating the MTBE produced, a mixture with the composition in accordance with Table II, column E is withdrawn via line 29 and supplied to the reactor 23, which is operated as described in Example 1. The composition of the product of line 36 is indicated in Table II, column F. Column G indicates the composition in line 39.

1. A process of producing C₂- and C₃-olefins from feed hydrocarbons, characterized in that the feed hydrocarbon together with steam is passed as vapor through a thermal steam cracking in which it is heated to temperatures in the range from 700 to 1000° C., where a cracking mixture is produced which contains C₂- to C₆-olefins and C₄- to C₆-diolefins, that a first fraction, which contains C₂- and C₃-olefins, and a second fraction, which contains olefins and diolefins of the range C₄ to C₆, are separated from the cracking mixture, that the diolefins are at least partly removed from the second fraction and an intermediate product is produced which consists of C₄-to C₆-olefins for at least 30 wt-%, that a feed mixture containing C₄- to C₆-olefins and steam is introduced into a reactor with an inlet temperature of 300 to 700° C., which reactor contains a bed of granular, form-selective catalyst, where a product mixture containing C₂- to C₄-olefins is withdrawn from the bed, and C₂- to C₃-olefins are separated from the product mixture.

2. The process as claimed in claim 1, characterized in that at least part of the second fraction is passed through a butadiene extraction.

3. The process as claimed in claim 1 or 2, characterized in that at least part of the second fraction is passed through a partial hydrogenation in which the diolefins are at least partly converted to olefins.

4. The process as claimed in claim 1 or any of the preceding claims, characterized in that at least part of the intermediate product which contains isobutene is introduced into a synthesis for producing methyl-tert-butylether (MTBE), and that the residual mixture coming from the synthesis is introduced into the reactor.
